(19) United States
(12) Patent Application Publication
(10) Pub. No.: US 2004/0243067 A1
(43) Pub. Date: Dec. 2, 2004

(54) Multidiameter syringe families

(76) Inventor: Wilmer L. Sibbitt JR., Albuquerque, NM (US)

Correspondence Address: ROBERTS ABOKHAIR & MARDULA, SUITE 1000, 11800 SUNRISE VALLEY DRIVE, RESTON, VA, 20191, US

(21) Appl. No.: 10/772,163

(22) Filed: Feb. 3, 2004

Related U.S. Application Data
(60) Provisional application No. 60/444,204, filed on Feb. 3, 2003.

(51) Int. Cl. A61M 5/315

(52) U.S. Cl. 604/219

(57) ABSTRACT

Danger from pricks by contaminated hypodermic needles is reduced by the configuring of a family of single diameter and multiple diameter syringes that accommodate conventional hypodermic needles but also a variety of universal needle-based guards, shields, and safety needles. The family of syringes has as a common property among all the members of the family—a standardized diameter of that portion of the syringe barrel adjacent the needle. The portion of the syringe barrels in the family having the standardized diameter can accommodate universal safety needles regardless of the volume of the syringe.

### CROSS REFERENCE TO RELATED APPLICATIONS

This application claims priority benefit under 35 U.S.C. § 119(e) of provisional application No. 60/444,204, filed Feb. 3, 2003, which is incorporated herein by reference.

### FIELD OF THE INVENTION

The present invention is directed to the art of medicators and receptors. More particularly, the present invention is directed to hypodermic syringes having selected diameters.

### BACKGROUND OF THE INVENTION

Hypodermic needles have been used for many years in industry, research, and medical practice. These needles, which consist of a hollow metal tube sharpened on one end in order to penetrate the skin or other substance, are attached to a syringe, which is used to aspirate or inject volumes of fluid through the hollow barrel of the needle.

Before use, the hypodermic needle is sharp but sterile. However, after use, the needle becomes contaminated with blood, body fluids, or residual fluid remaining from aspiration of samples from bottles. These contaminated hypodermic needles remain very sharp and easily penetrate the skin, directly depositing infectious agents into the body tissues of the medical worker.

Accidental penetration of the skin from sharp instruments is one of the most common modes of transmission of fatal or debilitating infectious diseases to health care workers. Hepatitis B, hepatitis C, and HIV (the AIDS virus) are typically transmitted in the health care environment from needle sticks and result in years of debilitating illness, loss of productivity, workman's compensation payments, medical expenses, and accelerated mortality. The groups of health care workers most susceptible to needle sticks include nurses and laboratory workers. However, physicians, dentists, dialysis workers, oral surgeons, medical waste workers, and animal handlers also have an elevated risk of exposure.

The Occupational Safety and Health Administration (OSHA) requires regular instruction of health care workers in techniques to prevent accidental needle sticks. Nevertheless, needle sticks from recapping syringes with needles and from uncapped or unsheathed syringe needles remain an important mechanism for transmission of virulent infectious agents to health care workers. Any advances in medical instrument design that would limit or prevent needle sticks would markedly reduce the health risks from infectious diseases for health care workers. This would result in considerable savings from lost productivity, medical costs, litigation, and compensation payments. Most importantly, the health and safety of health care workers would be improved.

One approach to this problem has been the use of guarded or shielded syringes or safety caps or shields that cover the syringe needle after use. These devices appear to be 70% effective in preventing needle sticks. However, many of the needle-based safety devices are limited in that they are specific to a given syringe size. Additionally, the syringe-based shields or guards increase the diameter of the syringe while at the same time decreasing the stability of the syringe platform. All of these negative factors discourage use of such syringes.

Guarded or shielded syringes have been demonstrated to markedly decrease needle stick injuries. However, such guarded or shielded syringes generally have features that make use of a syringe more difficult, thus, endangering the patient from an awkwardly yielded safety syringe. This is not acceptable to many operators. Thus, the safety shield is often removed or disabled, so that the syringe can be manipulated more easily. Alternatively, the operator may refuse to use this type of syringe and instead may choose a conventional syringe. This behavior, rooted in dissatisfaction with the ergonomics of a safety syringe, converts a guarded safety syringe into a conventional syringe, and ease of manipulation is gained, but the safety features are removed. Many institutions have thus instituted rules against disabling such safety devices, even though these safety devices are awkward and may endanger the patient by the operator using a clumsy, difficult device. This approach does not address the real problem, which is the lack of careful ergonomic considerations in the design of guarded syringes.

An additional concern is that safety devices are not universally applicable. Conventionally, safety devices tend to be syringe-specific. Thus, a separate model of safety device is required for each individual model of syringe. This increases inventory stocking headaches and tends to inhibit the widespread use of the safety devices.

### BRIEF SUMMARY OF THE INVENTION

One aspect of the present invention is that it provides for a family of syringe-devices with applications to health care, research, and industry. Upon further study of the detailed description infra and the appended claims, various advantages and objects of the invention will become apparent to those skilled in the art.

A family of syringes embodied according to the present invention includes both basic, otherwise conventional syringes with certain modifications made to be compatible with the family, as well as radically unconventional syringes, each with certain modifications made to be compatible with other the syringes in the family. Thus, the entire family of syringes, including those that are conventional and those that are unconventional, function together as a family. The family of syringes together and the individual syringe family members represent aspects of the present invention.

As will be readily understood by those skilled in the art, the danger from hypodermic needles is reduced by the configuring of a family of single diameter and multiple diameter syringes that accommodate conventional hypodermic needles but also a variety of universal needle-based guards, shields, and safety needles. One aspect of this invention is to provide a family of syringes that have as a common property, a standardized diameter of that portion of the syringe barrel adjacent the needle. The portion of the syringe barrels in the family having the standardized diameter can accommodate universal safety needles regardless of the volume of the syringe.

This standardization across a family of syringes may be implemented by modifying the conventional members of this syringe family to achieve this standard or lesser diameter. For larger volume members of the syringe family, the present invention is embodied by configuring the syringe barrel to have multiple diameters with a narrow (standardized diameter) end adjacent the needle. This narrowed, standard size diameter syringe barrel end accommodates a safety device close to the needle mounting and joined to the narrow diameter portion of the syringe barrel is a larger diameter portion of the barrel that accommodates the syringe volume required.

This modification (making a portion of the syringe barrel a standard, narrower diameter) permits safety needles or needle-based guards or shields to fit any syringe in the family. In this sense, the shields, guards, and safety needles become universal because they are interchangeable between syringes of various volumes.

As additional operational benefits, the narrowed barrel segment of the multiple diameter syringe permits the syringe (regardless of whether its diameters have been standardized or not) to be used in narrow operating fields, to approach a target at a more conducive angle, to permit the hands to be farther from operative site, to permit the use of shorter needles or medical instruments, and to permit easier filling of the syringe (in aspiration mode) from a bottle, a fluid container, or a body cavity.

Another additional benefit is that the use of multiple diameter syringes also enables economical syringe-based shields and guards, which are less awkward than conventional guarded or shielded syringes and maintain fine control of the syringe apparatus. Having the narrowed diameter portion of the barrel being a standardized size also permits the manufacture of the syringe-based guard to be interchangeable between the different syringe versions, markedly decreasing manufacturing costs.

This invention has wide applicability to hypodermic needles, which result in the bulk of sharp instrument injuries to medical workers.

### BRIEF DESCRIPTION OF THE DRAWINGS

FIG. 1 illustrates a side view of a plunger subassembly of a syringe according to a first embodiment of the present invention.

FIG. 2 illustrates a side view of a barrel subassembly of a syringe according to the first embodiment of the present invention.

FIG. 3 illustrates a partial section view of a syringe, according to the first embodiment of the present invention, in operation.

FIG. 4 illustrates a partial section view of a syringe, according to a second embodiment of the present invention, in operation.

FIG. 5 illustrates a side view of a plunger subassembly of a syringe according to a third embodiment of the present invention.

FIG. 6 illustrates a partial section view of a barrel subassembly of a syringe according to the third embodiment of the present invention.

FIG. 7 illustrates a partial section view of a syringe, according to the third embodiment of the present invention, in operation.

FIG. 8 illustrates a partial section view of a 1 ml syringe according to an exemplary family of syringes.

FIG. 9 illustrates a partial section view of a 3 ml or 5 ml syringe according to an exemplary family of syringes.

FIG. 10 illustrates a partial section view of a 5 ml or 10 ml syringe according to an exemplary family of syringes.

FIG. 11 illustrates a partial section view of a 10 ml, 20 ml, or 30 ml syringe according to an exemplary family of syringes.

FIG. 12 illustrates a partial section view of a 50 ml or 60 ml syringe according to an exemplary family of syringes.

FIG. 13 illustrates a partial section view of a shield for use with a family of syringes arranged according to any of the various embodiments of the present invention.

FIG. 14 illustrates a partial section view of the 1 ml syringe according to the exemplary family of syringes, with a shield in a retracted position.

FIG. 15 illustrates a partial section view of the 3 ml or 5 ml syringe according to the exemplary family of syringes, with a shield in an extended position.

FIG. 16 illustrates a partial section view of the 5 ml or 10 ml syringe according to the exemplary family of syringes, with a shield in a retracted position.

FIG. 17 illustrates a partial section view of the 10 ml, 20 ml, or 30 ml syringe according to the exemplary family of syringes, with a shield in a retracted position.

FIG. 18 illustrates a partial section view of the 50 ml or 60 ml syringe according to the exemplary family of syringes, with a shield in an extended position.

FIG. 19 illustrates a partial section view of a safety cap for use with a family of syringes arranged according to any of the various embodiments of the present invention.

FIG. 20 illustrates a partial section view of a syringe for use with a safety cap according to FIG. 19.

FIG. 21 illustrates a partial section view of a syringe without a shield and having a safety cap according to FIG. 19 mounted thereon.

FIG. 22 illustrates a partial section view of a syringe with a shield and having a safety cap according to FIG. 19 mounted thereon.

### DETAILED DESCRIPTION

The concept of a syringe family having standardized diameters and including multiple diameter syringes is a holistic vision of syringe design in which each individual syringe is a member of a larger and interrelated syringe family. Each member of an individual syringe family has a common property, that is, a segment of the syringe barrel is equal in diameter (length may be the same or may vary somewhat) between the different members of the family. This common property permits each member of an particular syringe family to be fit with safety devices that are universal in the sense that they are wholly interchangeable so as to fit on any member of the syringe family. The safety devices can be needle-based or syringe-based, and can be of various designs.

The innovations involved in the creation of these syringe families permit an institution to implement an overall syringe safety program in which the same universal safety devices will work across the entire line of syringes permitting cost savings and the general use of safety devices.

With a syringe family having standardized diameters and including multiple diameter syringes (i.e., a multidiameter syringe family) there is a manufacturing advantage, in that an individual safety device will fit each and every member of the syringe family, greatly reducing the costs of manufacturing safety devices and safety device systems. These new multidiameter syringe families will also permit the use of all conventional needles thus maintaining operator flexibility.

The syringes forming a multidiameter syringe family are multipurpose, and can completely supplant conventional syringes without surrendering flexibility while at the same time increasing safety and overall syringe ergonomics. It is anticipated that when an institution wishes to implement a universal safety system, they would buy the entire multidiameter syringe line, and completely dispense with conventional syringes.

On the other hand, a customer could buy individual members of the syringe family fitted with syringe-based safety devices, but the cost savings for the manufacturer would be maintained in that the same syringe-based safety device would fit any member of an individual syringe family. Thus, only one size of safety device need be manufactured because it would fit all of the syringes. This is a tremendous cost advantage for a manufacturer.

The design innovations used to create the multidiameter syringe families also produce individual syringes with greater utility, ergonomics, and specific uses, while maintaining their multipurpose properties. Thus, the larger members of a given syringe family have distinct practical advantages over their conventional counterparts in that the multidiameter syringes can be used in narrower operating fields, can approach a target at a more conducive angle, permit the hands to be farther from a dangerous operative site while permitting the use of a larger volume syringe, permit the use of shorter needles or medical instruments (which are more easily shielded). They also permit easier filling of the syringe (in aspiration mode) from a bottle, a fluid container, or a body cavity, and permit a more ergonomic use of a safety device so that it does not interfere with the use of a syringe.

Any of the syringes embodied according to the teachings of this invention may also include a reciprocating feature, without detracting in any way from the fundamental strength of the multidiameter syringe family concept. A reciprocating syringe has two or more plungers mechanically connected so that one plunger reciprocates with respect to the other plunger, enabling alternate injection or aspiration by changing only the thumb position.

The multidiameter syringe families concept is an important advance in syringe design that promotes universal safety precautions, maintains operator flexibility, permits use of conventional needles if desired, reduces safety device manufacturing costs, permits a manufacturer to market the entire syringe family line as an integrated “safety system,” and produces individual members of the syringe family that have special uses, yet maintains the flexibility provided in a mutually compatible syringe family.

Each syringe in a given family of syringes has a syringe barrel with flanges and a plunger with appropriate stoppers and thumb rest. The syringe barrel has a Luer, a Luer-Lok, or other fitting to connect to a hypodermic needle or other medical device. The syringes of a given family are related to one another by having a portion of the syringe barrel or an effective external portion of the syringe barrel having a diameter that is at or slightly less than a selected standard size. In some instances, the portion (or effective external portion) of the syringe barrel has a standardized length throughout the syringe family.

One way for individual syringes within a syringe family effect the standard diameter is to have a false barrel surround the real barrel.

Another way of implementing the standardization of diameter is for some syringes within the syringe family to be re-shaped to be longer or shorter than conventional syringes of the same volume.

For larger volume syringes, an effective way to accomplish the standardized diameter is to have a standard diameter narrow portion and a larger diameter non-standarized portion. The larger diameter portion accommodates the desired volume.

In this multiple diameter configuration, the plunger may be provided with a second stopper sized and positioned to empty the fluid from the narrow portion of the barrel. More specifically, if the internal diameter of the narrower standard diameter portion of the multiple diameter syringe is constant, then the plunger is modified to have a segment to move fluid in the narrow portion of the barrel and a segment to move fluid in the wider portion of the barrel. The segment of the plunger that moves fluid in the wider portion of the barrel is of a wider lower portion of the plunger with a first stopper at the end. The first stopper is sized for sealable and slidable engagement with the wider portion of the syringe barrel. The segment of the plunger that moves fluid in the narrow portion of the barrel is a narrow upper portion of the plunger with the second stopper at the end. The second stopper is sized for sealable and slidable engagement with the narrow portion of the syringe barrel. The plunger also has a channel, tunnel, grove, or other passage to permit movement of fluid between the narrow and wider compartments of the syringe barrel when the syringe is being operated for either aspiration or injection.

Alternatively, the second stopper may be omitted in favor of a single stopper plunger. If the internal diameter of the narrow, standardized portion of a multiple diameter syringe is tapering, an appropriate modification to the plunger is for the stopper to include a tapering segment for extending into the narrow, standardized portion of the barrel to move fluid in the narrow compartment.

One feature of a syringe family having standardized diameters and including multiple diameter syringes is that the syringes can be embodied so as to include shields in the form of extendable plastic cylinders (or other shape) that are capable of being extended from around the standard diameter narrow segment of the syringe barrel outwardly so as to cover a needle or other sharp. The shielded syringe advantageously has a locking or securing mechanism to stabilize the shield in the extended protective position.

It is useful to note that shields for use with such syringe families are standardized in diameter and/or length and, thus, can be used on any particular member of the syringe family.

Needle-based safety devices of various designs including, but not limited to, caps (with or without handles) are useful in combination with such a family of syringes, provided that the caps can fit over the diameter of the standard diameter portion of the syringe barrel or over the external diameter of a shield on the barrel. The caps advantageously are interchangeable between the different members of a given multidiameter syringe family.

As an example, a multiple diameter syringe according to various embodiments of the present invention has a barrel, a plunger movable inside the barrel, and a fluid conduit at one end of the barrel. The fluid conduit is adapted for a needle, catheter, or other similar structure to connect to the syringe. The barrel has a narrow barrel portion at the end of the barrel near the fluid conduit, and a wide barrel portion at the other end of the barrel. The wide barrel portion has a diameter that is substantially larger than the diameter of the narrow barrel portion. The plunger has a narrow plunger portion and a wide plunger portion. The narrow plunger portion is sized to move within the narrow barrel portion, and the wide plunger portion is sized to move within the wide barrel portion.

The multiple diameter syringes disclosed enable standardized or non-standardized syringes to be used in narrow operating fields, to approach a target at a more conducive angle, to permit the hands to be farther from operative site. They also enable the use of shorter needles or medical instruments.

The members of a syringe family may include one or more reciprocating syringes that have at least two plungers mechanically connected so that one plunger reciprocates with respect to the other plunger. This reciprocating structure enables use of the syringe to alternately inject or aspirate by changing only the position of the user's thumb.

A multidiameter syringe family integrated with universal safety devices forms a sharps safety system. Such a sharps safety system is appropriate for adoption by a health care institution to promote safer use of syringes.

Needle sticks from hypodermic needles occur in several ways:

accidental sticks that occur from improperly discarded uncapped needles on syringes,

accidental sticks that occur from the act of improperly discarding needles on syringes,

sticks that occur from attempts to recap the contaminated needle on a syringe,

sticks that occur to the operator from an uncapped needle not in immediate use, but still on the equipment tray or in the operating field,

sticks that occur in the operating field from a misdirected needle, or

intentional sticks.

The present invention is useful to decrease needle sticks occurring principally from improperly discarded uncapped needles on syringes, recapping injuries, and improper disposal of needles on syringes.

Although one aspect of the present invention is to provide a family of syringes that have the common property, that is, a syringe barrel portion diameter that is standardized so as to accommodate universal needle safety devices regardless of the volume of the syringe. The standard barrel diameter may be selected to be any diameter. Advantageously, the standard diameter is chosen to be in a range appropriate to accommodate the external diameter of a Luer-Lok device, or other common coupling. As a useful alternative, the standard diameter is chosen to be in a range appropriate to accommodate a Luer fitting. However, because of the risk of accidental dislodgment of a needle during a procedure, especially a procedure in which the syringe develops high-pressure, operators would expect the capability of a Luer-Lok. In this instance, the minimal diameter so as to accommodate a Luer-Lok is an appropriate selection for the standardized syringe diameter and is preferable over that to accommodate a Luer fitting. As a practical matter, since the Luer-Lok has a larger diameter than a Luer, to accommodate a Luer-Lok device, the external diameter of the narrow portion of the syringe is limited to the external diameter of the Luer-Lok device or larger. Accordingly, a salient aspect of the present invention is to select the standard diameter that will tend to accommodate any safety device.

Generally, though, a family of syringes could have any standard diameter selected to be compatible with a Luer fitting, a Luer-Lok, a catheter, or some specialized structures. All of structures fulfill useful purposes and are thus suitable choices on which to base a selection of standard diameter.

With the foregoing general guidance as to how to select an appropriate standard diameter, it has been found that standardized syringe barrel diameters in the range of from about 0.5 cm to about 3.0 cm are useful.

The members of a family of such syringes each provide a long narrow syringe barrel portion appropriate to accommodate mounting of a safety device and needles (conventional if desired, or otherwise). Optionally joined to this long narrow syringe barrel portion is a larger diameter barrel to accommodate the volume desired for that syringe. Because the syringe is composed of a barrel of either increased length to accommodate greater volume or a barrel with multiple external and internal diameters, plungers having a likewise modified configuration are appropriate.

The length of the long narrow portion may be selected in order to accommodate needles of a particular size. For example, if use of longer needles with the syringe family is desired, the length of the standardized portion of the barrel is expanded and the large portion is widened to accommodate greater volume.

These syringe modifications have a number of specific advantages independent of their compatibility with other members of a particular syringe family. For larger syringes, since the diameter of the barrel portion close the needle is narrower, the larger volume syringes can be used for fine procedures. That is because the diameter of the proximal portion of the syringe barrel does not interfere, either visually or physically, with close approach to the object. This will improve the technical performance of a number of syringe-based procedures; especially those used with larger syringes.

Similarly, in larger syringes, the stroke distance of the plunger will be decreased because the stroke distance will be determined by the length of the largest barrel portion. This will be an advantage with larger syringes where the stroke distance is long, as in a conventional 60 cc syringe. That is because it will be better suited for individuals with small hands who have difficulty accommodating the long stroke distance. It is also because the hand muscles have a greater mechanical advantage when the stroke distance of the plunger is less. Although the applied force may need to be greater to generate a greater pressure, but this is overcome by the mechanical advantage of the lesser stroke distance.

The basic design of the multidiameter syringe according to a first embodiment is a narrow barrel portion having a standardized diameter selected to accommodate the needle or safety device, and a larger barrel portion having variable length and variable diameter selectable to accommodate a desired fluid volume. One plunger accomplishes movement of fluid in the multidiameter syringe, thus, special consideration is given to the design of the plunger and stopper so that fluid can move freely between the upper narrow diameter syringe barrel portion and the lower wider diameter syringe barrel portion. This can be accomplished by have the internal diameter of the narrow barrel being constant and providing a groove or passage through the plunger to permit passage of fluid between the lower wider chamber and the upper narrower chamber. Some small amount of residual fluid will be trapped in the groove or passage in this design. To minimize the amount of residual fluid, the narrow portion of the plunger cannot have empty space; otherwise a substantial amount of fluid will be trapped within the syringe. If this is permitted to occur, the trapped fluid will prevent movement of both the plunger and the fluid. Accordingly, it is useful to have configurations that do not have dead space between the upper and lower portions of the plunger.

An example of a multiple diameter syringe according to the first embodiment of the present invention has a common property within a syringe family. The multiple diameter syringe has a barrel, a plunger movable inside the barrel, and a fluid conduit at one end of the barrel. The barrel has a narrow barrel portion at the end of the barrel near the fluid conduit, and a wide barrel portion at the other end of the barrel. The wide barrel portion has a diameter that is substantially larger than the diameter of the narrow barrel portion. The narrow barrel portion having a constant diameter interior profile. The plunger has a narrow plunger portion, a wide plunger portion, a wide resilient stopper, and a narrow resilient stopper. The narrow plunger portion is sized to move within the narrow barrel portion, and the wide plunger portion is sized to move within the wide barrel portion. The wide resilient stopper is located between the narrow plunger portion and the wide plunger portion, and is sized to slidably and sealably engage the inside surface of the wide barrel portion. The narrow resilient stopper is located on the narrow plunger portion, and is sized to slidably and sealably engage the inside surface of the narrow barrel portion. The narrow barrel portion has an outside diameter that is substantially equal to the standard diameter for the syringe family. A fluid flow channel is formed in the the narrow barrel portion so that fluid is free to flow through the fluid flow channel between a lower chamber formed inside the wide barrel portion and an upper chamber formed inside the narrow barrel portion.

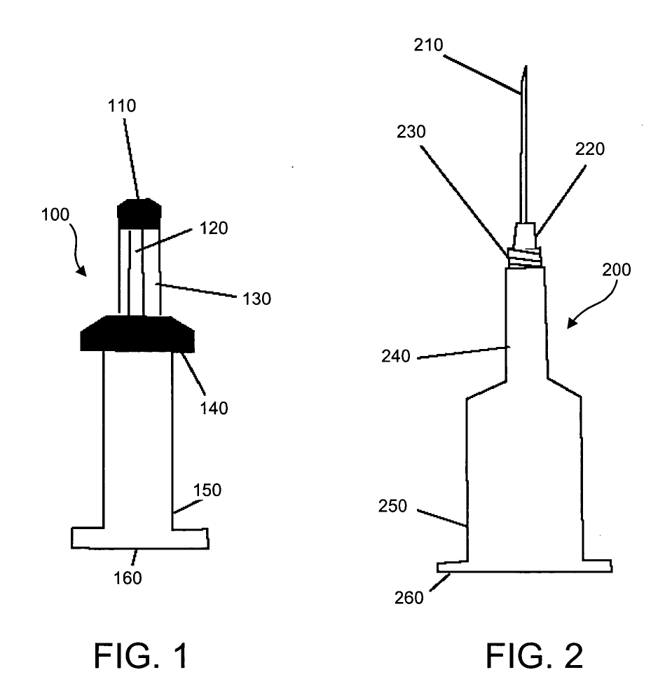

Referring to FIG. 1, a side view of a plunger subassembly of a syringe according to a first embodiment of the present invention is illustrated. The plunger 100 has a smaller upper rubber stopper 110 that can be discrete or continuous with plunger, a groove or tunnel 120 in the upper portion 130 of the plunger to permit movement of fluid through the upper portion 130 of plunger and move into the upper narrow portion of the barrel. The plunger also has a larger lower rubber stopper 140 that can be discrete or continuous with, and disposed between, the upper plunger portion 130 and the lower portion 150 of the plunger. A thumb rest 160 is disposed at the extreme end of the lower portion 150 of the plunger.

Referring to FIG. 2, a side view of a barrel subassembly of a syringe according to the first embodiment of the present invention is illustrated. The syringe barrel 200 has a hypodermic needle 210 (or optionally a cannula or nozzle) with a needle hub 220 with an integrated needle fitting 230. The needle fitting 230 may be a Luer, Luer-Lok, catheter or other similar, suitable structure. The syringe barrel 200 has an upper narrow barrel portion 240, a larger diameter lower barrel portion 250, and finger flanges 260.

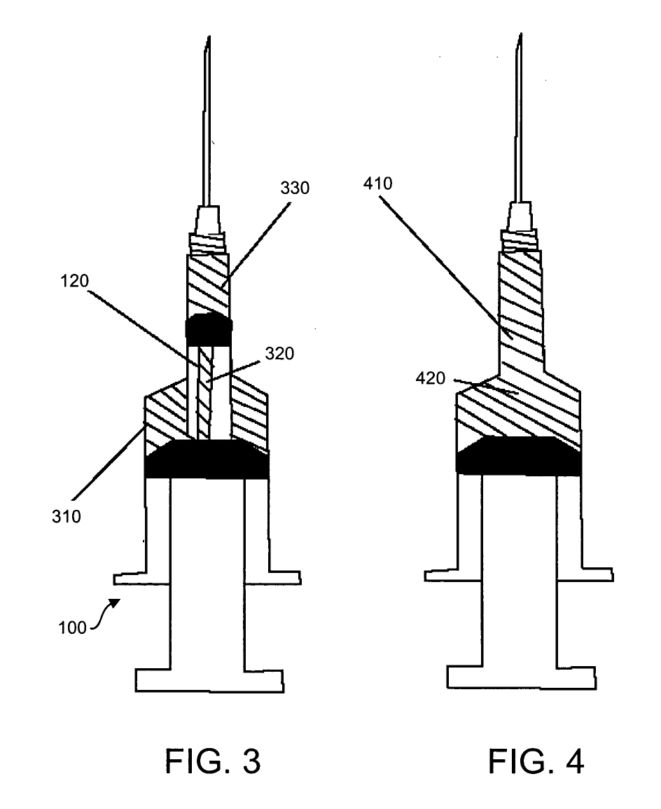

Referring to FIG. 3, a partial section view of a syringe, according to the first embodiment of the present invention, is illustrated as assembled and in operation. As the plunger 100 is depressed, fluid in the lower chamber 310 flows towards the upper chamber 330 via the grove or tunnel 120 in the upper plunger portion and moves 320 to join fluid in the upper chamber 330.

An example of a multiple diameter syringe according to a second embodiment of the present invention has a barrel, a plunger movable inside the barrel, and a fluid conduit at one end of the barrel. The barrel has a narrow barrel portion at the end of the barrel near the fluid conduit, and a wide barrel portion at the other end of the barrel. The wide barrel portion has a diameter that is substantially larger than the diameter of the narrow barrel portion. The plunger is sized to move within the wide barrel portion and has a resilient stopper located at an end of the stopper that is sized to slidably and sealably engage an inside surface of the wide barrel portion.

Referring to FIG. 4, a partial section view of a syringe, according to a second embodiment of the present invention, is illustrated in operation. This embodiment is similar to the first embodiment except that the plunger does not have a narrow upper plunger portion. Thus, some dead space is left in the narrow upper barrel 410 that harbors some amount of fluid that flows from the larger lower barrel 420 into the narrow upper barrel 410 cannot be cleared from the syringe. However, in larger syringes this volume may be negligible.

Although each syringe in a family can be utilized independently, the syringes within a family are configured so that a safety system is achievable when the family of multidiameter syringes is adopted by an institution so that universal safety devices are readily utilized on syringes.

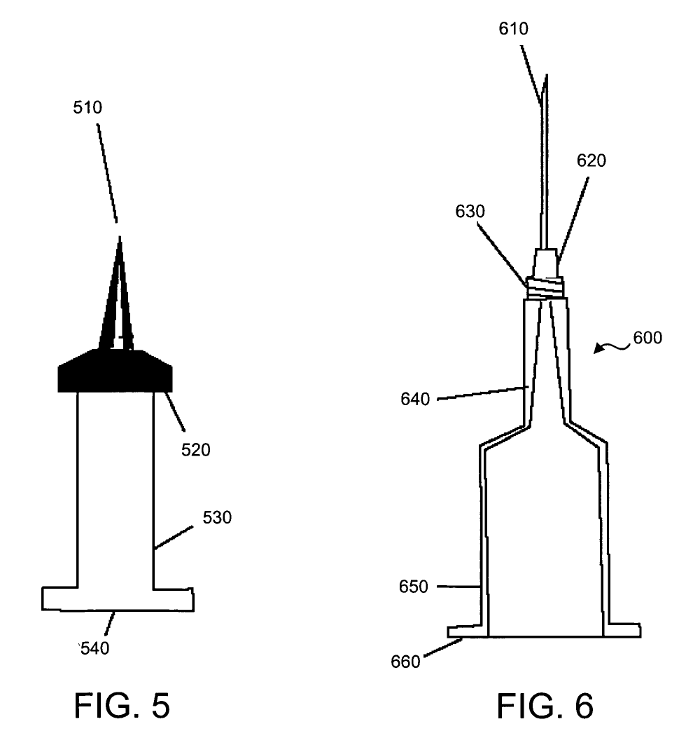

Referring to FIG. 5, a side view of a plunger subassembly of a syringe according to a third embodiment of the present invention is illustrated. The plunger has a rubber stopper with a tapered upper portion 510 and a wide lower portion 520. The rubber stopper upper portion 510 is tapered to permit movement of fluid along the plunger surface and to correspond to a tapered shape of the syringe barrel. The rubber stopper may be discrete or continuous with lower plunger 530. A thumb rest 540 is disposed at the bottom end of the lower plunger 530.

Referring to FIG. 6, a partial section view of a barrel subassembly of a syringe according to the third embodiment of the present invention is illustrated. A hypodermic needle 610 with a needle hub 620 is shown affixed to the barrel 600. Optionally, a cannula or nozzle may be affixed in place of a needle. The needle hub 620 connects to a needle fitting 630 at the end of the barrel 600. The needle fitting 630 is optionally configured as a Luer, Luer-Lok, catheter, or other type of fitting as needed. The barrel 600 has a narrow upper portion 640 having a tapered inner surface, and a larger diameter lower portion 650. Finger flanges 660 are disposed at the periphery of the lower end of the barrel 600. In this third embodiment, no groove or tunnel is necessary for fluid to move from the large diameter chamber to the upper chamber having a tapered diameter.

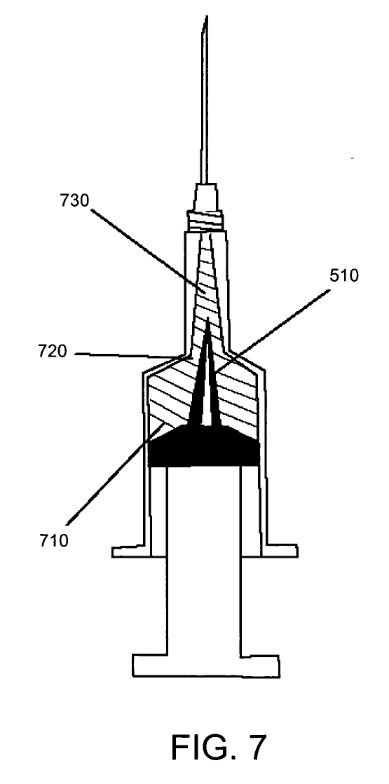

Referring to FIG. 7, a partial section view of a syringe, according to the third embodiment of the present invention, is illustrated in operation. As the plunger is depressed, fluid in the lower chamber 710 is forced by the wide lower portion 520 of the stopper to flow upward 720 past the tapered upper portion 510 of the stopper and moves to join fluid in the upper chamber 730.

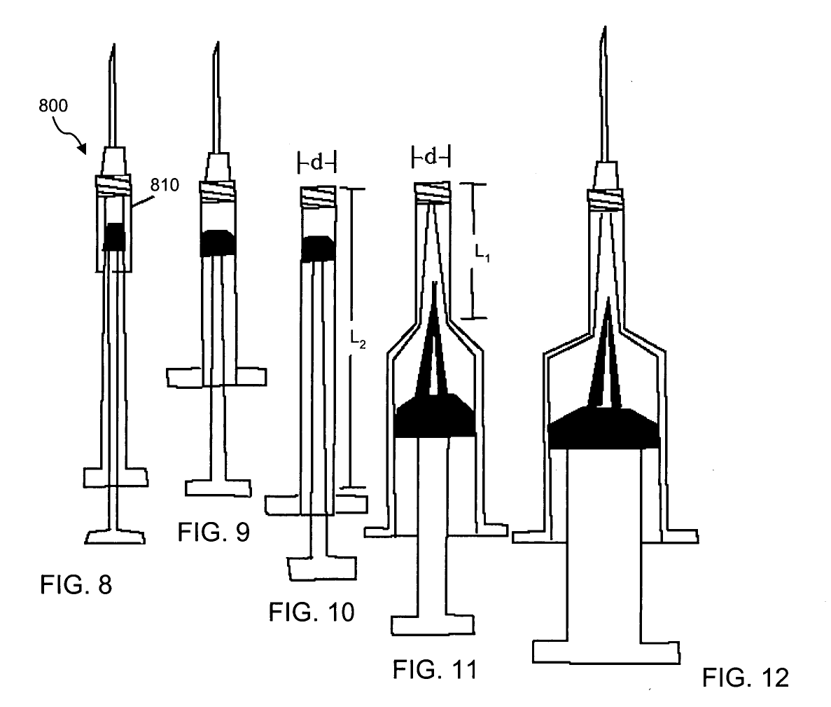

FIG. 8-12 portray an exemplary multidiameter syringe family. What unites the members of a syringe family is that they have an upper diameter that is the same as (or slightly less than) a standard diameter d. Because these devices have a common upper diameter d, they can accommodate a number of standard, interchangeable, or universal safety devices or needles as well as conventional needles.

Referring to FIG. 8, a partial section view of a 1 ml syringe is illustrated according to the exemplary family of syringes. For the 1 ml syringe 800, the upper diameter is narrower than the standard diameter d since for such a small volume barrel it is impractical to make it as wide as d. This discrepancy is accommodated by adding a false barrel 810 to the outside of the barrel. The false barrel 810 has a diameter of d.

A small volume syringe according to various embodiments of the present invention has a common property within a syringe family. The syringe has has a barrel, a plunger movable inside the barrel, a fluid conduit at one end of the barrel, and a resilient stopper located at the end of the plunger and sized to slidably and sealably engage an inside surface of the barrel. The barrel has an outside diameter that is less than a standard diameter of the syringe family. The syringe also has a false barrel surrounding the barrel and located at the end of the barrel with the fluid conduit. The false barrel has an outside diameter that is substantially equal to the standard diameter for the syringe family.

Referring to FIG. 9, a partial section view of a 3 ml or 5 ml syringe is illustrated according to the exemplary family of syringes. In certain of the syringes, particularly the 3, 5, and 10-ml versions, varying the length of the syringe, while maintaining the standard diameter d can accommodate the volume requirement. These longer versions can thus accommodate a larger safety device. Referring to FIG. 10, a partial section view of a 5 ml or 10 ml syringe according to the exemplary family of syringes is illustrated.

Referring to FIG. 11, a partial section view of a 10 ml, 20 ml, or 30 ml syringe according to the exemplary family of syringes is illustrated. For larger volumes the multidiameter syringe versions are required. Referring to FIG. 12, a partial section view of a 50 ml or 60 ml syringe according to an exemplary family of syringes is illustrated. The length L₁ of the upper narrow segment of the barrel determines the maximum length of the safety device for that syringe.

According to another embodiment of the present invention, a syringe family includes at least two syringes. Each of the syringes in the family has a different volume capacity that the other syringes. All of the syringes in the family share the common property of having a barrel that has an outside diameter at its needle end that is substantially equal to a standard diameter for the syringe family.

Reciprocating syringes may utilize this standardization concept to fit within a family of syringes. Reciprocating syringes have two or more plungers and one or more dependent or independent barrels, where the plungers are mechanically connected so that they alternate or reciprocate with each cycle of aspiration/injection.

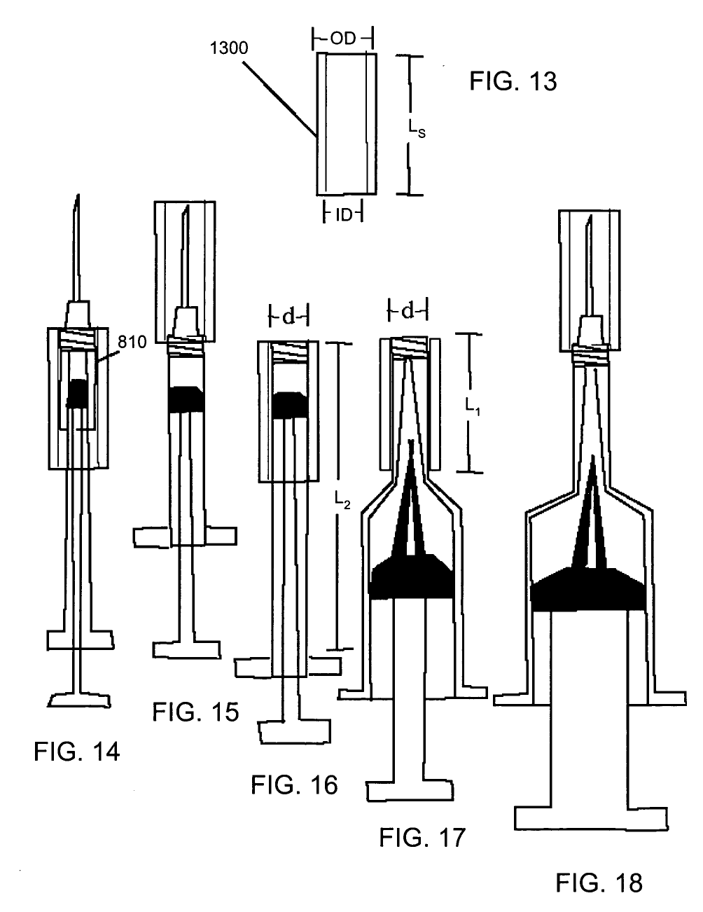

Referring to FIG. 13, a partial section view of a shield is illustrated. The shield is useful for integration with a family of syringes arranged according to any of the various embodiments of the present invention. The shield is an example of a standard guard or shield for the multidiameter syringe. Of course, practice of the present invention is not limited only to the shield shape illustrated and may take any number of modified configurations. The shield 1300 is composed of a plastic and has a length L_{S}. The shield 1300 has an inside diameter ID and an exterior diameter OD. The inside diameter ID of the shield 1300 is greater than or equal to the standard diameter d of the syringe and/or syringe family.

This shield 1300 optionally has intrinsic fasteners that lock the shield in a particular position, either extended or retracted. The locking devices may effect a position lock that is either permanent or temporary. Examples of suitable temporary or permanent locking devices are: 1) threaded or partially threaded components on the shield and syringe, 2) depressible tabs on the syringe which protrude and lock through fenestrations on the shield, 3) a series of angled and flexible retaining tabs facing opposite direction which would engage and trap an appropriate surface attached to the syringe, 4) a ring or partial ring composed of flexible material or rigid material with a spring or other material with memory that would engage an lock an appropriate retaining grove or space on the guard or shield or vice-versa. Other locking or fastening devices may also be utilized.

It would be anticipated that the length L_{S} of the shield 1300 would be roughly the same dimension as the lengths of narrow standardized portions of barrels of members of the syringe family (e.g., L₁), although it could be longer or shorter on individual syringes, and possibly as long as the maximum syringe length L₂.

This shield or guard could be designed specifically for each syringe size, or could be standardized and fit identically on any and all members of a particular syringe family. This shield could also be used in combination with a number of different needle based safety devices, as long as they could accommodate the outside diameter OD of the shield or the inside diameter ID. A standard shield that would fit on every member of a given syringe family has a salient manufacturing advantage.

Referring to FIG. 14, a partial section view of the 1 ml syringe, according to the exemplary family of syringes, is illustrated with a shield in place. On the 1 ml syringe the inside diameter ID may be made to correspond to the intrinsic diameter of the syringe but to fit compatibly on the other various members of the syringe family the inside diameter ID is selected to correspond to the size of the false barrel 810. The shield is shown in a retracted positions on the 1 ml syringe.

Referring to FIG. 15, a partial section view of the 3 ml or 5 ml syringe, according to the exemplary family of syringes, is illustrated with a shield in place. This shield (or guard) is shown in an extended position on the 3 or 5 mil syringe.

Referring to FIG. 16, a partial section view of the 5 ml or 10 ml syringe, according to the exemplary family of syringes, is illustrated with a shield in place. The shield is shown in a retracted positions on the 5 or 10 ml syringe.

Referring to FIG. 17, a partial section view of the 10 ml, 20 ml, or 30 ml syringe, according to the exemplary family of syringes, is illustrated with a shield in place. The shield is shown in a retracted position on the 10, 20, or 30-ml syringe.

Referring to FIG. 18, a partial section view of the 50 ml or 60 ml syringe, according to the exemplary family of syringes, is illustrated with a shield in place. The shield is shown in an extended position on the 50 or 60 ml syringe.

Needle-based safety devices are useable on these multidiameter syringes and syringe families with or without the shield, although the presence of the shield would naturally affect the design and dimensions of many of the needle-based safety devices.

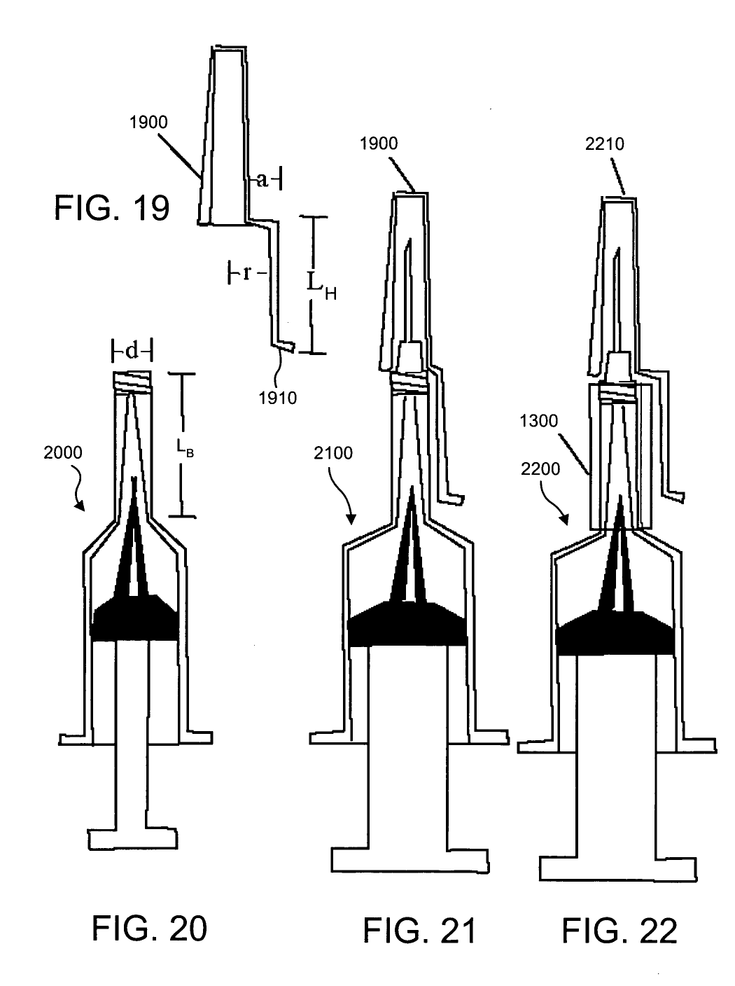

Referring to FIG. 19, a partial section view of a safety cap is illustrated. The safety cap is useful for integration with a family of syringes arranged according to any of the various embodiments of the present invention. The safety cap 1900 is an example of a needle based safety device. The safety cap shown has a handle 1910. However, the diameter of the medical device that this type of cap can accommodate is limited to a length that is twice the distance r from the central axis of the safety cap to the handle. The length of the handle L_{H} also places a limit on dimensions of the medical device that this type of cap can accommodate.

Referring to FIG. 20, a partial section view of a syringe is illustrated for use with a safety cap according to FIG. 19. In terms of the multidiameter syringe 2000, the value of two times r must at least be equal or greater than the external diameter d of the standardized portion of the syringe, and the length of the handle L_{H} is limited by the length L_{B} of the standardized portion of the barrel of the syringe 2000. Once the safety device fulfills these constraints it can fit on any member of a given syringe family.

Referring to FIG. 21, a partial section view of a syringe is illustrated without a shield and having a safety cap according to FIG. 19 mounted thereon. A safety device designed to fit on the multidiameter syringe with a shield will naturally also fit on non-shielded versions 2100 of these syringes. This safety cap device 1900 is shown only as an example. Many other safety devices of many designs could be standardized to fit interchangeably on an entire family of these shielded or non-shielded multidiameter syringes.

Referring to FIG. 22, a partial section view of a syringe is illustrated with a shield and having a safety cap according to FIG. 19 mounted thereon. Once a shielded version 2200 of this syringe is used however, the safety device parameter of r is adjusted to accommodate the external diameter of the standard shield 1300, in this case by lengthening the offset dimension a of the handle. Such a modified safety device 2210 is seated on a shield syringe 2200 as shown.

Although the present invention has been described in terms of a number of exemplary embodiments, it will be understood by those of skill in the art that various improvements, modifications, and simplifications may be made to the embodiments disclosed without departing from the scope of the invention.

What is claimed is:

1. A multiple diameter syringe comprising:
a barrel;
a fluid conduit in fluid communication with a proximal end of the barrel; and
a plunger disposed inside the barrel;
wherein the barrel comprises:
a narrow barrel portion adjacent the proximal end of the barrel, and
a wide barrel portion adjacent a distal end of the barrel, the wide barrel portion having a diameter that is substantially larger than the diameter of the narrow barrel portion; and
wherein the plunger comprises:
a narrow plunger portion sized to move within the narrow barrel portion, and
a wide plunger portion sized to move within the wide barrel portion.

2. The multiple diameter syringe of claim 1, wherein the plunger further comprises:
a wide resilient stopper disposed between the narrow plunger portion and the wide plunger portion, the wide resilient stopper being sized to slidably and sealably engage an inside surface of the wide barrel portion.

3. The multiple diameter syringe of claim 2, wherein the narrow barrel portion has a constant diameter interior profile and wherein the plunger further comprises:
a narrow resilient stopper disposed on the narrow plunger portion, the narrow resilient stopper being sized to slidably and sealably engage an inside surface of the narrow barrel portion.

4. The multiple diameter syringe of claim 3, wherein a fluid flow channel is formed in the the narrow barrel portion so that fluid is free to flow through the fluid flow channel between a lower chamber formed inside the wide barrel portion and an upper chamber formed inside the narrow barrel portion.

5. The multiple diameter syringe of claim 2, wherein the narrow barrel portion has a tapered interior profile and wherein the narrow plunger portion comprises:
a tapered stopper having a shape that corresponds substantially to the tapered interior profile of the narrow barrel portion.

6. The multiple diameter syringe of claim 1, further comprising:
a shield in slidable engagement with the narrow barrel portion.

7. A multiple diameter syringe having a common property within a syringe family, the multiple diameter syringe comprising:
a barrel;
a fluid conduit in fluid communication with a proximal end of the barrel; and
a plunger disposed inside the barrel;
wherein the barrel comprises:
a narrow barrel portion adjacent the proximal end of the barrel, the narrow barrel portion having a constant diameter interior profile, and
a wide barrel portion adjacent a distal end of the barrel, the wide barrel portion having a diameter that is substantially larger than the diameter of the narrow barrel portion;
wherein the plunger comprises:
a narrow plunger portion sized to move within the narrow barrel portion,
a wide plunger portion sized to move within the wide barrel portion,
a wide resilient stopper disposed between the narrow plunger portion and the wide plunger portion, the wide resilient stopper being sized to slidably and sealably engage an inside surface of the wide barrel portion, and
a narrow resilient stopper disposed on the narrow plunger portion, the narrow resilient stopper being sized to slidably and sealably engage an inside surface of the narrow barrel portion;
wherein narrow barrel portion has an outside diameter that is substantially equal to a standard diameter for the syringe family; and
wherein a fluid flow channel is formed in the the narrow barrel portion so that fluid is free to flow through the fluid flow channel between a lower chamber formed inside the wide barrel portion and an upper chamber formed inside the narrow barrel portion.

8. A multiple diameter syringe comprising:
a barrel;
a fluid conduit in fluid communication with a proximal end of the barrel; and
a plunger disposed inside the barrel;
wherein the barrel comprises:
a narrow barrel portion adjacent the proximal end of the barrel, and
a wide barrel portion adjacent a distal end of the barrel, the wide barrel portion having a diameter that is substantially larger than the diameter of the narrow barrel portion; and
wherein the plunger is sized to move within the wide barrel portion and the plunger comprises:
a resilient stopper disposed at a proximal end of the plunger, the resilient stopper being sized to slidably and sealably engage an inside surface of the wide barrel portion.

9. The multiple diameter syringe of claim 8, further comprising:
a shield in slidable engagement with the narrow barrel portion.

10. A syringe having a common property within a syringe family, the syringe comprising:
a barrel having an outside diameter that is less than a standard diameter of the syringe family;
a fluid conduit in fluid communication with a proximal end of the barrel; and
a plunger disposed inside the barrel;
a resilient stopper disposed the proximal end of the plunger sized to slidably and sealably engage an inside surface of the barrel;
a false barrel surrounding the barrel and disposed at the proximal end of the barrel, the false barrel having an outside diameter that is substantially equal to the standard diameter for the syringe family.

11. The syringe of claim 10, further comprising:
a shield in slidable engagement with the proximal end of the barrel.

12. A syringe family comprising:
two or more syringes, wherein each of the syringes has a different volume capacity that the other syringes, and wherein all of the syringes share the common property of having a barrel that has an outside diameter at its proximal end that is substantially equal to a standard diameter for the syringe family.

13. The syringe family of claim 12, wherein at least one of the two or more syringes has a barrel comprising:
a narrow barrel portion adjacent the proximal end of the barrel, and
a wide barrel portion adjacent a distal end of the barrel, the wide barrel portion having a diameter that is substantially larger than the diameter of the narrow barrel portion.

14. The syringe family of claim 13, wherein at least one of the two or more syringes has a barrel that has an outside diameter that is less than a standard diameter of the syringe family, wherein a false barrel surrounds the proximal end of the barrel, the false barrel having an outside diameter that is substantially equal to the standard diameter for the syringe family.
